(19) United States
(12) Patent Application Publication
(10) Pub. No.: US 2016/0082293 A1
(43) Pub. Date: Mar. 24, 2016

(54) HIFU TREATMENT OPTIMIZATION IN VICINITY OF SENSITIVE ZONES

(71) Applicant: KONINKLIJKE PHILIPS N.V., Eindhoven (NL)

(72) Inventors: ILPO ASKO JULIUS KOSKELA, Eindhoven (NL); KIRSI ILONA NURMILAUKAS, EINDHOVEN (NL); JAAKKO JUHANI TOLO, EINDHOVEN (NL); REKO TAPIO VUORINEN, EINDHOVEN (NL)

(21) Appl. No.: 14/889,472

(22) PCT Filed: May 8, 2014
(86) PCT No.: PCT/EP2014/059400

§ 371 Date: Nov. 6, 2015

(30) Foreign Application Priority Data
May 8, 2013 (EP) 13167031.7

(51) Int. Cl. A61N 7/02

(52) U.S. Cl. CPC A61N 7/02

(57) ABSTRACT

The present invention provides a method for heating a target zone (3) of a subject of interest (1) according to pre-defined heating requirements using ultrasonic irradiation, comprising the steps of providing an ultrasonic irradiation device comprising a set of individually controllable transducer elements in vicinity of the target zone (3), defining at least one sensitive zone (2) within an area (4) covered by ultrasonic irradiation device, and controlling the ultrasonic irradiation device to apply sonications of ultrasonic energy to the target zone (3) to achieve the desired heating thereof, wherein the transducer elements are individually controlled in phase and amplitude to provide the sonications as a beam (5) directed towards the target zone (3), wherein the beam (5) has a energy distribution so that the pre-defined heating requirements of the target zone (3) are met and the exposure of the at least one sensitive zone (2) is minimized The present invention further provides an ultrasonic irradiation device adapted to perform the above method. By individually controlling the transducer elements, beam shaping of the ultrasonic irradiation can be applied over sensitive zones like scars, bones, bowels, spines or others without associating an intensity limit or energy exposure limit thereto. Such a configuration of active elements is sought, that the exposure on the sensitive zone is minimized, without compromising focal properties or violating restrictions on the number of active elements.

### FIELD OF THE INVENTION

The invention relates to the field of High Intensity Focused Ultrasonic Treatments of a subject of interest.

### BACKGROUND OF THE INVENTION

Focused ultrasound systems are used for delivering sonications of acoustic energy into a tissue region within a patient to coagulate or otherwise treat the tissue region with thermal energy. Typical applications of ultrasonic heating are the treatment of cancerous or benign tumors, which are located in the treated tissue region, also referred to as target zone. For example, a piezoelectric transducer located outside the patient's body may be used to focus high intensity acoustic waves, such as ultrasonic waves (acoustic waves with a frequency greater than about twenty kilohertz (20 kHz)), in an internal tissue region of the patient to treat the internal tissue region. The acoustic waves may be used to ablate a tumor, thereby eliminating the need for invasive surgery. Such focused ultrasound systems are in particular provided as systems for High Intensity Focused Ultrasonic (HIFU), also referred to as HIFU devices. Pulsed heat is generated by the HIFU device, which is positioned to selectively destroy tissue of the patient with minimal invasiveness. In order to concentrate the sonications within the target zone, the sonications are provided as a beam to the target zone. Additionally, by employing magnetic resonance (MR) imaging apparatus, fast scan MR images can be used to monitor the tissue temperature with a temperature sensitive pulse sequence.

A focused ultrasound system known from U.S. Pat. No. 8,002,076 B2 includes a transducer array with a number “n” of individually controllable transducer elements, a driver, a controller, and a switch. The transducer array delivers acoustic energy into a target region, e.g., a benign or malignant tumor or other tissue volume, within a patient's body, to ablate or otherwise treat tissue within the target region. The switch connects the transducer array to the driver and the controller in order to steer and/or focus the acoustic energy transmitted by the transducer array in a desired manner. The ultrasound transducer concentrates heat at its focus which is positioned on the tumor. When delivering acoustic energy, it is known to control the shape of a “focal zone”, which corresponds to the volume of tissue treated when the acoustic energy is focused into a tissue region, as well as to control “focal depth”, which is a distance from the transducer to the focal zone, and/or to correct for tissue aberrations that may be caused by intervening tissue between the transducer and the tissue region.

In therapy using HIFU systems, the volume which can be treated is often limited by the presence of sensitive tissues in the near field, which are for example, scars, bones, bowels, or in the far field, which are for example spines or bowels, in direction of the acoustic path. The term “near field” corresponds to an area between the transducer and the target zone, and the term “far field” corresponds to an area further away from the transducer than the target zone.

For example, in the treatment of uterine fibroids, one is often concerned of scars and bowels in the near field, and the spine and bowels in the far field. There is a need to protect sensitive tissues from undesired exposure to ultrasound. In particular, the sensitive tissues are tissue types with high ultrasonic absorptivity, as well as tissue interfaces with high reflectivity. Excessive absorption of ultrasound in such regions may lead to undesired heating and undesired damage of tissue, which is not to be treated by the HIFU.

As known from U.S. Pat. No. 8,002,076 B2, if an analysis discovers that there are sensitive tissues, e.g. obstructions or high sensitivity volumes, along an energy pass zone when performing the sonications, in which it is desired to prevent acoustic energy from passing, e.g., air-filled cavities, non-targeted thick bone, and the like, individual transducer elements may be deactivated, e.g., have their amplitude set to zero, in order to prevent acoustic energy from being transmitted by the relevant transducer elements. Accordingly, sonications can be restricted to such positions which are deemed safe.

This approach has its inherent limitations. First, the volume than can be treated is limited. Second, the power loss due to the missing elements must be compensated by increasing the power of remaining elements, increasing the related surface heating. Moreover, switching off too many elements distorts the focal shape and, eventually, the achieved thermal dose. As a consequence of specifying sensitive zones and associating intensity/energy exposure limits with them, it typically becomes impossible to sonicate from certain transducer positions. This limits the achievable treatment volume, i.e. the size of the target zone. Typically, the necrotic region is elongated towards the transducer. Finally, the usefulness of the method decreases rapidly towards the focus, since all element beams are passing through the same regions.

### SUMMARY OF THE INVENTION

It is an object of the invention to provide a method and a device for heating a target zone of a subject of interest according to pre-defined heating requirements using ultrasonic irradiation, which enable the treatment of a target zone in vicinity of a sensitive zone, even if the sensitive zone is located between the transducer and the target zone, which provide an efficient heating of the target zone, and which reduce the risk of damages to tissues of sensitive zones, which have a high ultrasonic absorptivity and/or which have a tissue interface at their border with high reflectivity.

This object is achieved by a method for heating a target zone of a subject of interest according to pre-defined heating requirements using ultrasonic irradiation, comprising the steps of providing an ultrasonic irradiation device comprising a set of individually controllable transducer elements in vicinity of the target zone, defining at least one sensitive zone within an area covered by ultrasonic irradiation device, and controlling the ultrasonic irradiation device to apply sonications of ultrasonic energy to the target zone to achieve the desired heating thereof, wherein the transducer elements are individually controlled in phase and amplitude to provide the sonications as a beam directed towards the target zone, wherein the beam has an energy distribution so that the pre-defined heating requirements of the target zone are met and the exposure of the at least one sensitive zone is minimized.

This object is also achieved by an ultrasonic irradiation device for heating a target zone of a subject of interest using ultrasonic irradiation according to pre-defined heating requirements, comprising a set of individually controllable transducer elements to be located in vicinity of the target zone, and a control unit for controlling the application of sonications of ultrasonic energy to the target zone to achieve the desired heating thereof, wherein the control unit is adapted to receive a definition of at least one sensitive zone within an area covered by ultrasonic irradiation device, and the control unit is further adapted to individually control the transducer elements in phase and amplitude to provide the sonications as a beam directed towards the target zone, wherein the beam has an energy distribution so that the pre-defined heating requirements of the target zone are met and the exposure of the at least one sensitive zone is minimized.

By individually controlling the transducer elements, beam shaping of the beam of ultrasonic irradiation can be applied over sensitive zones like scars, bones, bowels, spines or others without associating an intensity limit or energy exposure limit thereto. Accordingly, a beam with a given shape can be provided, which has internally an inhomogeneous energy distribution, i.e. different parts of the beam can have a different energy level of ultrasonic irradiation. Such a configuration of active elements is sought, that the exposure on the sensitive zone is minimized, without compromising focal properties or violating restrictions on the number of active elements. It is merely required that in no part of the sensitive zone to be protected the intensity or energy is raised above the level that there would be without beam shaping, i.e. when the transducer is operated without taking care about the sensitive zone. Preferably, a set of active transducer elements that gives the smallest maximum or average intensity or energy inside the sensitive zone is automatically determined. Accordingly, the control first focuses on the heating of the target zone according to the pre-defined heating requirements, and within this focus, the exposure of the at least one sensitive zone is minimized. Hence, the treatment can be efficiently performed, and at the same time the sensitive zone is protected. For example, when scars define a sensitive zone, it is practically impossible to determine optimal exposure limits. Scars vary from subject to subject, and often sonication through the scar would be clinically feasible, although not recommended by state of the art methods. Hence, often the state of the art methods deny sonications, which would be actually feasible. According to the above embodiment, sonication through scars becomes feasible while gradual protection is still offered.

The beam can have any desired shape for performing the treatment. Preferably, the beam has a conical shape, where the tip of the cone is preferably located within the target zone. Alternatively, the beam can have a tubular shape.

The ultrasonic irradiation device can be provided for internal or external application of sonications in respect to the subject of interest. Accordingly, the ultrasonic irradiation device can be positioned on the skin of the subject of interest, or internally, e.g. when being introduced through any existing opening of the subject of interest. A device for external application of sonications is e.g. known as HIFU device (High Intensity Focused Ultrasonic device).

The pre-defined heating requirements may comprise any suitable definition of the required treatment, e.g. in terms of a thermal dose, tissue temperature and/or energy. The target zone is defined in the ultrasonic irradiation device prior to the treatment. Although a zone/area is frequently referred to as a 2-dimensional object, it is to be noted that in the context of this documents a zone/area objects having a 2-dimensional or 3-dimensional extension.

The term vicinity refers to any location that enables a desired heating of the target zone, e.g. on the skin of the subject of interest, or internally. In general, the saller the distance between the ultrasonic irradiation device and the target zone, the easier is the heating of the target zone. Further preferred, an ultrasonically conductive contact medium is provided between the ultrasonic irradiation device and the subject of interest, e.g. when the ultrasonic irradiation device is positioned on the skin of the subject of interest.

The ultrasonic irradiation device may have any suitable design. It may comprise individual units for different tasks, or different units of the ultrasonic irradiation device may be adapted to perform different tasks. In particular, the ultrasonic irradiation device may comprise a data processing unit. The control unit controls the transducer elements to perform the sonications. It may perform control of further components of the ultrasonic irradiation device. The control unit may be further adapted to perform additional tasks in the context of the application of ultrasonic irradiation. Preferably, the control unit comprises a data processing unit. The control unit comprises an interface for receiving the definition of the at least one sensitive zone. The interface may be a user interface for interacting with a user, or any other electronic interface for electronically receiving the definition of the at least one sensitive zone.

According to a preferred embodiment the control unit is adapted to adjust the amplitude of each transducer element over a continuous range between zero and a maximum amplitude value. In the corresponding method, the step of controlling the transducer elements individually in phase and amplitude comprises adjusting the amplitude of each transducer element over a continuous range between zero and a maximum amplitude value.

According to a preferred embodiment the control unit is adapted to adjust the phase of each transducer element over a continuous range of phase values. In the corresponding method, the step of controlling the transducer elements individually in phase and amplitude comprises adjusting the phase of each transducer element over a continuous range of phase values.

As specified above, each transducer element can be individually controlled by performing an adjustment over a continuous range, i.e. in case of the amplitude, any amplitude between zero and a maximum amplitude value of the transducer element can be set. This continuous adjustment enables an accurate control to meet the pre-defined heating requirements of the target zone and to minimize the exposure of the at least one sensitive zone.

According to a preferred embodiment the control unit is adapted to receive a diagnostic image at least partially covering the area covered by ultrasonic irradiation device, and the control unit is further adapted to identify the at least one sensitive zone within the diagnostic image. In the corresponding method, the step of defining at least one sensitive zone within an area covered by ultrasonic irradiation device comprises receiving a diagnostic image at least partially covering the area covered by ultrasonic irradiation device, and identifying the at least one sensitive zone within the diagnostic image. The diagnostic image can be used prior to starting the treatment to accurately define sensitive zones. Furthermore, the diagnostic image can be used to identify a sensitive zone, e.g. if the sensitive zone is positioned inside the subject of interest. The diagnostic image can be provided as a MR scan using a magnetic resonance imaging device, or by any other suitable method for providing diagnostic images. The diagnostic image can be provided as 2-dimensional image, as a set of 2-dimensional images, or as a 3-dimensional image.

According to a preferred embodiment the ultrasonic irradiation device comprises a simulation unit for simulating the sonications of ultrasonic energy from the transducer elements of the ultrasonic irradiation device to the area covered by ultrasonic irradiation device to achieve the desired heating of the target zone, and a visualization unit for visualizing a result of the simulation performed by the simulation unit indicating a level of protection of the at least one sensitive zone. The corresponding method comprises the additional step of simulating the sonications of ultrasonic energy from the transducer elements of the ultrasonic irradiation device to the area covered by ultrasonic irradiation device to achieve the desired heating of the target zone, and visualizing a result of the simulation step indicating a level of protection of the at least one sensitive zone. The simulation prior to the treatment enables a prediction, if the treatment in the presence of the at least on sensitive zone is feasible and should be performed. Preferably, based on the simulation result, a user can choose between different treatments, i.e. between different ways to control the transducer elements. Accordingly the user can select the treatment to be applied based on the most suitable simulation result. The simulation is preferably based on a diagnostic image of the area covered by ultrasonic irradiation device, which enables a prediction of the heating of the area covered by ultrasonic irradiation device due to knowledge about the tissues in this area. The result of the simulation can be visualized by colors or contours representing power intensity or energy. The visualization can also be done by visualizing the sensitive zone in different ways, e.g. representing the amount of gained intensity or energy reduction, which is visualized for example by shading or coloring the different zones within the area covered by ultrasonic irradiation device. Preferably, the system visualizes to the user the achieved protection level inside the sensitive zone. Further preferred, this is visualized by colors or contours representing power intensity or energy, inside and outside the sensitive zone. The simulation unit may be provided integrally with the control unit, or as a separate unit. The visualization unit may comprise any kind of display for visualizing the result of the simulation.

According to a preferred embodiment the ultrasonic irradiation device is adapted to display a numeric value indicative of the level of protection of the at least one sensitive zone on the visualization unit. In the corresponding method the step of visualizing a result of the simulation step indicating a level of protection of the at least one sensitive zone comprises displaying a numeric value indicative of the level of protection of the at least one sensitive zone. For example the maximum simulated intensity or energy inside the at least one sensitive zone with the chosen optimized control of transducer elements can be displayed as a percentage of the maximum intensity or energy that would be inside the sensitive zone without beam shaping.

According to a preferred embodiment the ultrasonic irradiation device is adapted to visualize the level of protection of the at least one sensitive zone as a relative level of protection of the sensitive zone compared to an area outside the sensitive zone on the visualization unit. In the corresponding method the step of visualizing a result of the simulation step indicating a level of protection of the at least one sensitive zone comprises visualizing the level of protection of the at least one sensitive zone as a relative level of protection of the sensitive zone compared to an area outside the sensitive zone. The relative level of protection can be specified in terms of gained intensity or energy reduction inside and outside the sensitive zone.

According to a preferred embodiment the control unit is adapted to receive temperature information of at least a part of the area covered by ultrasonic irradiation device, and the control unit is adapted to individually control the transducer elements by comparing the temperature information to the pre-defined heating requirements. The corresponding method comprises the additional step of providing temperature information of at least a part of the area covered by the ultrasonic irradiation device, and wherein the step of individually controlling the transducer elements in phase and amplitude, so that the pre-defined heating requirements are met and the exposure of at least one sensitive zone is minimized, comprises individually controlling the transducer elements by comparing the temperature information to the pre-defined heating requirements. With the knowledge of the temperature information, the heating of the target zone as well as the exposure of the sensitive zone can be determined to adapt the control of the ultrasonic irradiation device. In particular, the heating of sensitive zones is difficult to predict, so that they can be efficiently monitored to minimize their exposure to the sonications. Preferably, the temperature information is obtained by performing a magnetic resonance scan. The control unit may comprise an electronic interface for receiving the temperature information in any suitable format.

According to a preferred embodiment the ultrasonic irradiation device comprises a deflection unit, and the control unit is adapted to perform a control of the deflection unit and/or the transducer elements to deflect the sonications of ultrasonic energy from the ultrasonic irradiation device to the target zone to minimize the heating of the at least one sensitive zone. In the corresponding method, the step of individually controlling the transducer elements in phase and amplitude, so that the pre-defined heating requirements are met and the exposure of at least one sensitive zone is minimized, comprises deflecting the sonications of ultrasonic energy from the ultrasonic irradiation device to the target zone to minimize the heating of the at least one sensitive zone. Accordingly, the ultrasonic irradiation can at least partially be directed to the target zone without passing through the sensitive zone. Without deflection, there can be a large overlap between the beam of the ultrasonic irradiation and the sensitive zone. By using deflection, the overlap can be reduced while still targeting the same focal point.

According to a preferred embodiment the deflection unit is an electronic deflection unit for electronically deflecting the sonications. In the corresponding method, the step of deflecting the sonications of ultrasonic energy from the ultrasonic irradiation device to the target zone comprises electronically deflecting the sonications. This can be easily applied to perform the deflection of the sonications. Preferably, electronic deflection is chosen based on the criteria that acoustic exposure on the sensitive zone is not exceeded.

According to a preferred embodiment the control unit is adapted to control the transducer elements to perform volumetric sonications. In the corresponding method, the step of individually controlling the transducer elements in phase and amplitude, so that the pre-defined heating requirements are met and the exposure of at least one sensitive zone is minimized, comprises controlling the transducer elements to perform volumetric sonications. In single-point sonication, the focal point of the ultrasonic irradiation is kept at fixed position, and the thermal dose is controlled through the applied power and the duration of the sonication. Volumetric sonications comprise of a series of rapidly interleaved single-point sonications, which on the time scale of thermal diffusion appear as simultaneous. Therefore, when performing volumetric sonication, the focal point is moved along a planned trajectory, distributing the heating across the desired volume. Preferably, this is achieved by a combination of using tranducers having phased arrays of transducer elements and electric deflection. The volumetric sonications are chosen based on the criteria that acoustic exposure on the sensitive zones is not exceeded. This maximizes the treatable volume without endangering the sensitive zone. This can be applied either in near-field, to protect scars or other sensitive tissues, or in far-field, to protect the spine and/or bowels. An advantage of volumetric sonications is that they are more energy-effective than single-point sonications, so that more volume can be treated for the same amount of energy.

According to a preferred embodiment the control unit is adapted to select volumetric cells from a list of predefined volumetric cells, and the control unit is further adapted to locate the volumetric cells within the target zone. In the corresponding method, the step of controlling the transducer elements to perform volumetric sonications comprises forming volumetric cells within the target zone based on the shape of the target zone and/or the shape and position of the at least one sensitive zone, and locating the volumetric cells within the target zone. The predefined cells can have any shape, e.g. spherical, ellipsoid, cubical or others. Hence, the volumetric cells can be easily applied for each treatment enable treatments with a low preparation time. The control unit can automatically select the volumetric cells and locate them within the target zone, or it can be adapted to receive a selection of the volumetric cells and their locations. The control unit can also have a user interface for interacting with a user to select and locate the volumetric cells.

According to a preferred embodiment the control unit is adapted to form volumetric cells within the target zone based on the shape of the target zone and/or the shape and the position of the at least one sensitive zone, and the control unit is further adapted to locate the volumetric cells within the target zone. In the corresponding method, the step of controlling the transducer elements to perform volumetric sonications comprises forming volumetric cells on the target zone and/or the information on the at least one sensitive zone, and locating the volumetric cells within the target zone. Hence, the volumetric cells can be adapted to each treatment individually to achieve the best heating of the target zone. The control unit can automatically form the volumetric cells and locate them within the target zone, or it can be adapted to receive a definition of the volumetric cells and their locations. The control unit can also have a user interface for interacting with a user to define and locate the volumetric cells.

### BRIEF DESCRIPTION OF THE DRAWINGS

These and other aspects of the invention will be apparent from and elucidated with reference to the embodiments described hereinafter. Such an embodiment does not necessarily represent the full scope of the invention, however, and reference is made therefore to the claims and herein for interpreting the scope of the invention.

In the drawings:

FIG. 1 shows a schematic illustration of a subject of interest and an ultrasonic beam of an ultrasonic irradiation device together with a deflected beam.

### DETAILED DESCRIPTION OF EMBODIMENTS

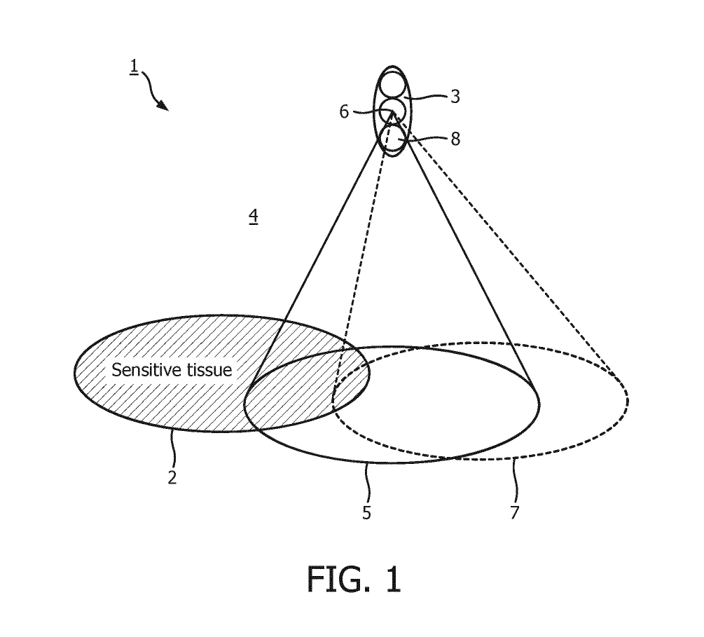

FIG. 1 shows a schematic illustration of a subject of interest 1 having a sensitive zone 2, which is a scar in this embodiment. Furthermore, a zone of cancerous tissue can be seen in the FIG., which corresponds to a target zone 3 for treatment with an ultrasonic irradiation device, which is HIFU device in this embodiment. The HIFU device is not shown in the FIG. The target zone 3 is heated according to pre-defined heating requirements using ultrasonic irradiation from the HIFU device. The pre-defined heating requirements in this embodiment refer to a thermal dose to be applied to the target zone 3 to ablate the tissue within the target zone 3.

The sensitive zone 2 is defined within an area 4 covered by ultrasonic irradiation device based on a diagnostic image, which is in this embodiment a 3-dimensional MR scan provided using a magnetic resonance imaging device. The diagnostic image covers the area 4 covered by ultrasonic irradiation device, which includes the target zone 3 and sensitive zone 2. Target zone 3 and sensitive zone 2 are identified and defined within the diagnostic image prior to starting the treatment. The area 4 covered by ultrasonic irradiation device, the target zone 3 and the sensitive area 2 have a 3-dimensional extension. The definition of the target zone 3 and the sensitive area 2 are provided to a control unit of the HIFU device.

The ultrasonic irradiation device in this embodiment is provided for external application of sonications in respect to the subject of interest 1 and is positioned on the skin of the subject of interest 1 in this embodiment. The area 4 covered by ultrasonic irradiation device, the target zone 3 and the sensitive area 2 are located inside the subject of interest 1, i.e. below the skin. The ultrasonic irradiation device is located close to the target zone 3, i.e. in vicinity to the target zone 3, so the distance between the target zone 3 and the ultrasonic irradiation device is short and only a low power loss of the irradiation occurs between the target zone 3 and the ultrasonic irradiation device.

The ultrasonic irradiation device comprises a set of individually controllable transducer elements and is controlled to apply sonications of ultrasonic energy to the target zone 3 to achieve the desired heating thereof. The transducer elements are individually controlled to adjust the amplitude over a continuous range between zero and a maximum amplitude value and the phase over a continuous range of phase values, so that the pre-defined heating requirements of the target zone 3 are met, i.e. in case of the amplitude, any amplitude between zero and the maximum amplitude value of each transducer element can be set individually. This control enables to provide the sonications as a beam 5, which is directed towards the target zone 3. This control further enables beam shaping, wherein the beam 5 has an energy distribution so that the pre-defined heating requirements of the target zone 3 are met and the exposure of the sensitive zone 2 is minimized. Accordingly, a beam 5 with a given shape is provided, which internally has an inhomogeneous energy distribution, i.e. different areas of the beam 5 have a different energy level of ultrasonic irradiation. The control is performed by the control unit.

In this embodiment, the sonications of ultrasonic energy from the ultrasonic irradiation device to the target zone 3 are electronically deflected to further minimize the heating of the sensitive zone 2. This is achieved by directing the beam 5 of ultrasonic irradiation to the target zone 3 without passing through the sensitive zone 2, as can be seen in the FIG. The beam 5 of ultrasonic irradiation, which has a focal point 6 within the target zone 3, has an overlap with the sensitive zone 2. Accordingly, the beam 5 is deflected, as can be seen from the deflected beam 7, so that the focal point 5 is the same, but the overlap with the sensitive zone 2 is reduced to almost zero. The HIFU device comprises an electronic deflection unit for deflecting the ultrasonic irradiation, which is controlled by the control unit. The energy distribution within the beam 5 is chosen so that in the remaining overlap of the deflected beam 7 with the sensitive zone 2 is reduced compared to the part of the deflected beam 7 not overlapping with the sensitive zone 2.

In this embodiment the transducer elements are further controlled by the control unit to perform volumetric sonications. In single-point sonication, the focal point 6 of the ultrasonic irradiation is kept at fixed position, and the thermal dose is controlled through the applied power and the duration of the sonication. Volumetric sonications comprise a series of consecutive single-point sonications. Accordingly, when performing volumetric sonication, the focal point 6 is moved along a planned trajectory, distributing the heating across the desired volume. This control is achieved by a combination of the individual control and adjustment of the transducer elements in respect to continuous values of phase and amplitude in combination with deflection as described above. The volumetric sonications are chosen based on the criteria that acoustic exposure on the sensitive zone 2 is not exceeded. As can be seen in the FIG., in this embodiment three volumetric cells 8 having a spherical shape are selected from a list of predefined volumetric cells and located within the target zone 3 to define the volumetric sonications.

During the treatment, MR scans of the area 4 are provided containing temperature information of the area 4 covered by ultrasonic irradiation device, i.e. the sonication zone. The temperature information is compared by the control unit to the thermal dose to continuously adapt the control of the transducer elements during the treatment.

In this embodiment, the sonications of ultrasonic energy from the transducer elements of the ultrasonic irradiation device to the area 4 covered by ultrasonic irradiation device to achieve the desired heating of the target zone 3 are simulated prior to starting the treatment in a simulation unit of the HIFU device. The result of the simulation is visualized using a visualization unit of the HIFU device to indicate a level of protection of the sensitive zone 2. Based on the simulation result, a user chooses between different treatments, i.e. between different ways to control the transducer elements. The simulation is based on the diagnostic image of the area 4 covered by ultrasonic irradiation device. The result of the simulation step is visualized by colors representing ultrasonic energy, whereby the amount of energy reduction is visualized by coloring the different zones 2, 3 within the area 4 covered by ultrasonic irradiation device. Additionally, numeric values indicative of the level of protection are visualized, which display a percentage of the maximum energy that would be inside the sensitive zone 2 without beam shaping.

Overall, the transducer elements are individually controlled in amplitude and phase, so that the exposure on the sensitive zone 2 is minimized, without compromising focal properties or violating restrictions on the number of active elements. It is merely required that in no part of the sensitive zone 2 the intensity or energy is raised above the level that there would be without beam shaping, i.e. when the transducer is operated without taking care about the sensitive zone 2. The set of active transducer elements that gives the smallest maximum or average energy inside the sensitive zone 2 is automatically determined in the simulation step.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### REFERENCE SYMBOL LIST

1 subject of interest
2 sensitive zone
3 target zone
4 area covered by ultrasonic irradiation device
5 beam
6 focal point
7 deflected beam
8 volumetric cell

1. An ultrasonic irradiation device for heating a target zone of a subject of interest using ultrasonic irradiation according to pre-defined heating requirements, comprising:
a set of individually controllable transducer elements
a deflection unit for deflecting the ultrasonic irradiation
a control unit for controlling the application of sonications of ultrasonic energy to the target zone to achieve a desired heating of the target zone, wherein the control unit is adapted to receive a definition of at least one sensitive zone within an area covered by ultrasonic irradiation device, and the control unit is further adapted to individually control the transducer elements in phase and amplitude to provide the sonications as a beam directed towards the target zone, wherein the beam has an internal inhomogeneous energy distribution that is controlled by the phase and amplitude of the transducer elements, so that pre-defined heating requirements of the target zone are met and the exposure of the at least one sensitive zone is minimized and
the control unit is adapted to perform a control of the deflection unit and/or the transducer elements to deflect the sonications of ultrasonic energy from the ultrasonic irradiation device to the target zone to minimize the heating of the at least one sensitive zone.

2. The ultrasonic irradiation device of claim 1, wherein
the control unit is adapted to adjust the amplitude of each transducer element over a continuous range between zero and a maximum amplitude value.

3. The ultrasonic irradiation device of claim 1, wherein the control unit is adapted to adjust the phase of each transducer element over a continuous range of phase values.

4. The ultrasonic irradiation device of claim 1, wherein the control unit is adapted to receive a diagnostic image at least partially covering the area covered by ultrasonic irradiation device, and
the control unit is adapted to identify the at least one sensitive zone within the diagnostic image.

5. The ultrasonic irradiation device of claim 1, comprising
a simulation unit for simulating the sonications of ultrasonic energy from the transducer elements of the ultrasonic irradiation device to the area covered by ultrasonic irradiation device to achieve the desired heating of the target zone, and
a visualization unit for visualizing a result of the simulation performed by the simulation unit indicating a level of protection of the at least one sensitive zone.

6. The ultrasonic irradiation device of claim 5, wherein
the ultrasonic irradiation device is adapted to display a numeric value indicative of the level of protection of the at least one sensitive zone on the visualization unit.

7. The ultrasonic irradiation device of claim 5, wherein
the ultrasonic irradiation device is adapted to visualize the level of protection of the at least one sensitive zone as a relative level of protection of the sensitive zone compared to an area outside the sensitive zone on the visualization unit.

8. The ultrasonic irradiation device of claim 1, wherein
the control unit is adapted to receive temperature information of at least a part of the area covered by ultrasonic irradiation device, and
the control unit is adapted to individually control the transducer elements by comparing the temperature information to the pre-defined heating requirements.

9. (canceled)

10. The ultrasonic irradiation device of claim 1, wherein
the deflection unit is an electronic deflection unit for electronically deflecting the sonications.

11. The ultrasonic irradiation device of claim 1, wherein
the control unit is adapted to control the transducer elements to perform volumetric sonications.

12. The ultrasonic irradiation device of claim 11, wherein
the control unit is adapted to select volumetric cells from a list of predefined volumetric cells, and
the control unit is further adapted to locate the volumetric cells within the target zone.

13. The ultrasonic irradiation device of claim 11, wherein
the control unit is adapted to form volumetric cells within the target zone based on shape of the target zone and/or the shape and position of the at least one sensitive zone, and
the control unit is further adapted to locate the volumetric cells within the target zone.

14. A method for heating a target zone of a subject of interest according to pre-defined heating requirements using ultrasonic irradiation, comprising the steps of
providing an ultrasonic irradiation device comprising a set of individually controllable transducer elements in vicinity of the target zone,
defining at least one sensitive zone within an area covered by ultrasonic irradiation device,
deflecting the ultrasonic irradiation by a deflection unit and
controlling the ultrasonic irradiation device to apply sonications of ultrasonic energy to the target zone to achieve the desired heating thereof, wherein the transducer elements are individually controlled in phase and amplitude to provide the sonications as a beam directed towards the target zone, wherein the beam has an internal inhomogeneous energy distribution that is controlled by phase and amplitude of the transducer elements so that the pre-defined heating requirements of the target zone are met and the exposure of the at least one sensitive zone is minimized and
control of the deflection unit and/or the transducer elements to deflect the sonications of ultrasonic energy from the ultrasonic irradiation device to the target zone to minimize the heating of the at least one sensitive zone.
